(19) United States
(12) Patent Application Publication
(10) Pub. No.: US 2025/0174955 A1
(43) Pub. Date: May 29, 2025

(54) SYNCHRONIZATION FOR LASER PULSE CONTROL

(71) Applicant: Alcon Inc., Fribourg (CH)

(72) Inventor: David Jung, Tustin, CA (US)

(21) Appl. No.: 18/931,366

(22) Filed: Oct. 30, 2024

Related U.S. Application Data
(60) Provisional application No. 63/603,743, filed on Nov. 29, 2023.

(51) Int. Cl. H01S 3/00

(52) U.S. Cl. CPC H01S 3/0057; H01S 3/0007

(57) ABSTRACT

A laser system includes a laser. The laser is configured to emit electromagnetic radiation in laser pulses. The laser system includes an optical switching device configured to switch between a first condition that allows laser pulses emitted by the laser to be output from the laser system and a second condition that prevents the laser pulses emitted by the laser from being output from the laser system. The laser system includes a pulse picking controller configured to receive inputs from an input device and communicate optical switching control signals to the optical switching device based on the inputs. The pulse picking controller selects at least one of a synchronous firing timer or an asynchronous firing timer. The asynchronous firing timer causes a beginning of a pulse picking cycle to start based on receipt of an input of the inputs.

### BACKGROUND

Lasers are used in many different medical procedures including a number of different ophthalmic procedures. For example, lasers may be used in cataract surgery, such as for fragmenting the cataractous lens. In some procedures, a laser is used for initial fragmentation of the lens, followed by phacoemulsification of the lens by an ultrasonic handpiece to complete the breakdown of the lens for removal. In other procedures, the laser may be used for complete fragmentation and/or phacoemulsification of the lens for removal, without the need for a separate application of ultrasonic energy. Lasers may also be used for other steps in cataract surgery, such as for making corneal incision(s) and/or opening the capsule.

Lasers may also be used in glaucoma surgery. For example, a laser may be used to form all or part of a channel through the trabecular meshwork or scleral tissue for drainage of aqueous humor from the eye.

Lasers may also be used in vitreoretinal surgery. In some procedures, a laser may be used for vitrectomy, to sever or break the vitreous fibers for removal. The laser may be incorporated into a vitrectomy probe, and the energy from the laser may be applied to the vitreous fibers to sever or break the vitreous fibers for removal.

In other vitreoretinal applications, lasers may be used for photocoagulation of retinal tissue. Laser photocoagulation may be used to treat issues such as retinal tears and/or the effects of diabetic retinopathy.

### SUMMARY

Aspects of the present disclosure relate to surgical systems for ophthalmic (eye) procedures, and more specifically, to laser systems for ophthalmic procedures.

In certain embodiments, a laser system for ophthalmic procedures is provided. The laser system includes a laser, an optical switching device, and a pulse picking controller. The laser is configured to emit electromagnetic radiation in laser pulses. The optical switching device is configured to switch between a first condition that allows laser pulses emitted by the laser to be output from the laser system and a second condition that prevents the laser pulses emitted by the laser from being output from the laser system. The pulse picking controller is configured to receive inputs from an input device, communicate optical switching control signals to the optical switching device based on the inputs, and select at least one of a synchronous firing timer or an asynchronous firing timer. The asynchronous firing timer causes a beginning of a pulse picking cycle to start based on receipt of an input of the inputs.

In certain embodiments, a method includes emitting laser pulses by a laser of a laser system, receiving inputs from an input device, controlling an optical switching device based on the inputs, and selecting at least one of a synchronous firing timer or an asynchronous firing timer. The laser is configured to emit electromagnetic radiation in laser pulses. The optical switching device is configured to switch between a first condition that allows the laser pulses emitted by the laser to be output from the laser system and a second condition that prevents the laser pulses emitted by the laser from being output from the laser system. The asynchronous firing timer is configured to cause a beginning of a pulse picking cycle to start based on receipt of an input of the inputs.

### BRIEF DESCRIPTION OF THE DRAWINGS

The drawings described herein are for illustrative purposes only, are schematic in nature, and are intended to be exemplary rather than to limit the scope of the disclosure.

FIG. 1 illustrates an example surgical console of a surgical system, according to embodiments described herein.

FIG. 2 illustrates an example surgical system including a laser system, according to embodiments described herein.

FIG. 3 illustrates an example architecture of a pulse picking controller, according to embodiments described herein.

FIG. 4 illustrates an example of a message to communicate as an input to a pulse picking controller, according to embodiments described herein.

FIG. 5 illustrates an example of operational subranges for an input device, according to embodiments described herein.

FIG. 6 illustrates an example representation of selecting an asynchronous firing timer, according to embodiments described herein.

FIG. 7 illustrates an example flow diagram for selecting a synchronous firing timer or an asynchronous firing timer, according to embodiments described herein.

The above summary is not intended to represent every possible embodiment or every aspect of the subject disclosure. Rather the foregoing summary is intended to exemplify some of the novel aspects and features disclosed herein. The above features and advantages, and other features and advantages of the subject disclosure, will be readily apparent from the following detailed description of representative embodiments and modes for carrying out the subject disclosure when taken in connection with the accompanying drawings and the appended claims.

### DETAILED DESCRIPTION

Aspects of the present disclosure relate to surgical systems for ophthalmic procedures, and more specifically, to laser systems for ophthalmic procedures.

The designations “first” and “second” as used herein are not meant to indicate or imply any particular positioning or other characteristic. Rather, when the designations “first” and “second” are used herein, they are used only to distinguish one component from another. The terms “attached,” “connected,” “coupled,” and the like mean attachment, connection, coupling, etc., of one part to another either directly or indirectly through one or more other parts, unless direct or indirect attachment, connection, coupling, etc., is specified.

As described above, laser systems are used in many different ophthalmic procedures. Some of these laser systems emit laser beams in pulses, with the pulses having a desired duration and repetition rate. Operating a laser in pulses can achieve desirable power and energy characteristics for a particular application. In addition, while the energy of a beam emitted by a laser can be controlled by controlling the laser source itself, in some systems it is desirable to control the amount of energy of a laser beam downstream from the laser source. Existing systems for laser pulse selection typically have one or more drawbacks, such as power loss, complexity, cost, etc. There is a need for improved systems and methods for laser pulse control.

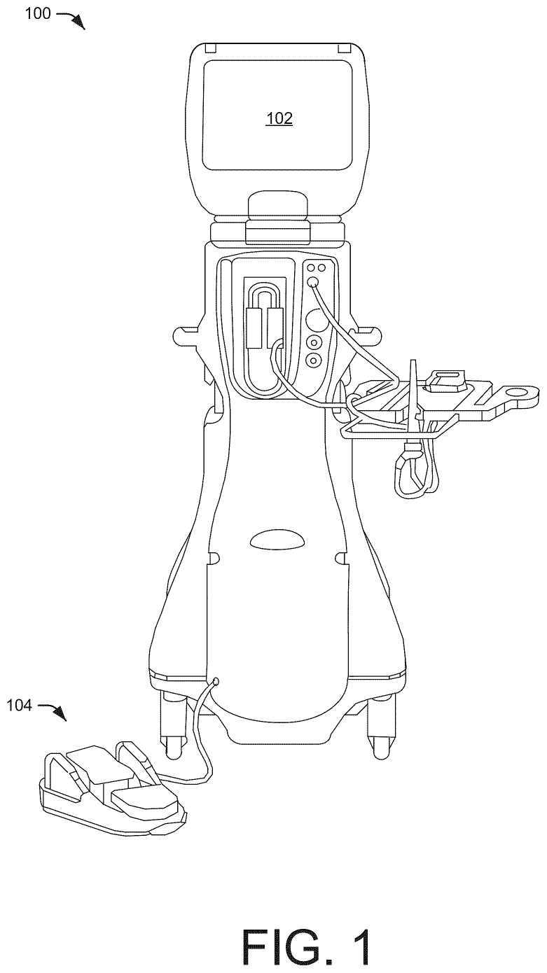

FIG. 1 illustrates an example surgical console of a surgical system, according to embodiments described herein. As depicted in FIG. 1, the example ophthalmic surgical console 100 includes a housing and a display device 102. The housing contains one or more computing systems, which control the functionality of one or more systems of the surgical console 100. For example, the surgical console 100 may include a fluidics system that includes an irrigation system for delivering fluid to an eye and an aspiration system for aspirating fluid from the eye. The display device 102 may display information relating to system operation and performance during an ophthalmic surgical procedure. The display device 102 can also include a user interface such that interactions with user interface elements of the user interface cause the one or more computer systems to modify functionality provided by at least one system of the surgical console 100 that may be used in performing an ophthalmic surgical procedure.

The surgical system also includes an input device 104 which is illustrated as a footswitch. The input device 104 can be communicatively coupled to the surgical console 100 via a wired or wireless connection. The input device 104 may be an adjustable input device that an operator may actuate over an operating range for controlling one or more functions. In the illustrated example in which the input device 104 is a footswitch, the footswitch can be pressed downward to various positions over the operating range to control functionality as described further below. While a footswitch is shown, the input device 104 can include other adjustable input devices, such as hand-operated buttons or knobs. An example surgical system in accordance with this disclosure may include a laser system suitable for one or more ophthalmic procedures.

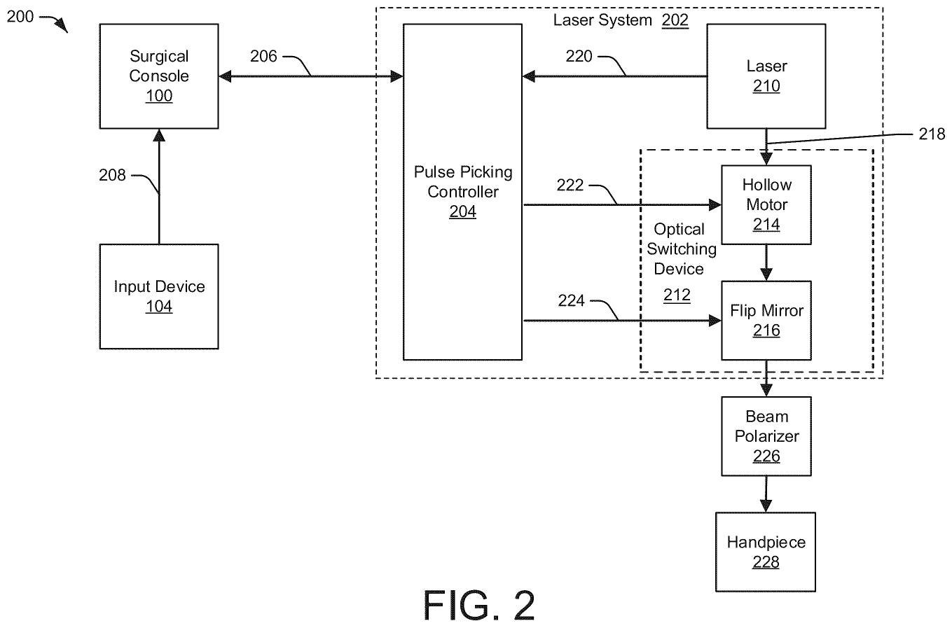

FIG. 2 illustrates an example surgical system 200 including a laser system, according to embodiments described herein. As shown, example surgical system 200 includes a surgical console 100, an input device 104, and an example laser system 202. In various embodiments, the laser system 202 includes a pulse picking controller 204, which is communicatively coupled to the surgical console 100 via a connection 206 that can be a wired connection or a wireless connection. The pulse picking controller 204 may receive information (e.g., inputs or messages) transmitted from the surgical console 100 via the connection 206, and the pulse picking controller 204 can also transmit information (e.g., confirmations) to the surgical console 100 via the connection 206. In some embodiments, the input device 104 is communicatively coupled to the surgical console 100 via a wired or wireless connection 208. For example, the input device 104 may transmit information (e.g., inputs) to the surgical console 100 via the connection 208.

In various embodiments, the laser system 202 may include a laser 210 and an optical switching device 212. In some embodiments, the laser system 202 may be housed within the surgical console 100. In other embodiments, the laser system 202 may be housed in a separate console that communicates with the surgical console 100. In some other embodiments, one or more parts of the laser system 202, such as the laser 210 and the optical switching device 212, may be housed in a separate console that communicates with the surgical console 100, and one or more other parts of the laser system 202, such as the pulse picking controller 204, may be housed in the surgical console 100. In other embodiments, the laser system 202 may be in a stand-alone housing that receives inputs from the input device 104 without the need for a separate surgical console 100.

The optical switching device 212 is illustrated to include a hollow motor 214 and a flip mirror 216. In some embodiments, the hollow motor 214 may be a rotary motor, such as a rotary servo motor, which is configured to control a rotation of various optical elements such as mirrors, lenses, prisms, etc. that can be included in the optical switching device 212. For example, the various optical elements may be attached to a rotatable element such as a shaft, and the hollow motor 214 controls the rotation of the various optical elements by rotating the rotatable element. By controlling the rotation of the various optical elements, the hollow motor 214 can direct (or aim) a laser pulse train 218 without physically moving the laser system 202.

In various embodiments, the flip mirror 216 includes an optical switch (e.g., a rotatable mirror, such as a galvo mirror) that is capable of directing the laser pulse train 218 between different optical pathways. In some embodiments, the flip mirror 216 controls the optical switch to switch between a first state and a second state. In the first state, the flip mirror 216 may allow the laser pulse train 218 to be output from the laser system 202. In the second state, the flip mirror 216 can prevent the laser pulse train 218 from being output from the laser system 202.

In one or more embodiments, the pulse picking controller 204 receives a laser trigger 220 from the laser 210. In some embodiments, the pulse picking controller 204 may process the laser trigger 220 to control an amount of electromagnetic radiation included in laser pulses output from the laser system 202 via a laser power control 222. The hollow motor 214 can include an attached waveplate which is an optical device that alters a polarization state of light (e.g., the laser pulse train 218) that passes through the waveplate. For instance, altering the polarization state of light by blocking particular electromagnetic waves and passing specific electromagnetic waves can control the amount of electromagnetic radiation included in the light. In some examples, the pulse picking controller 204 causes the hollow motor 214 to rotate the waveplate via the laser power control 222 in order to control the amount of electromagnetic radiation included in the laser pulses output from the laser system 202.

In various embodiments, the pulse picking controller 204 can process the laser trigger 220 to control timing and other characteristics (e.g., frequency, duty ratio, etc.) of the laser pulses output from the laser system 202 via a pulse picking control 224. In some embodiments, the pulse picking controller 204 controls the optical switch of the flip mirror 216 via the pulse picking control 224 to perform passing and blocking of laser pulses (e.g., the laser pulse train 218). By selectively passing and blocking the laser pulses, the pulse picking controller 204 may control the timing and the other characteristics of the laser pulses output from the laser system 202.

In one or more embodiments, a beam polarizer 226 operates in conjunction with the waveplate of the hollow motor 214 to pass or block electromagnetic radiation in an amount controlled by the rotation of the waveplate. For example, light that is polarized (e.g., the laser pulse train 218) passes through the waveplate of the hollow motor 214 which rotates the light that is polarized based on the rotational position of the waveplate. In an example, the waveplate of the hollow motor 214 rotates the light that is polarized between 0 to 90 degrees based on the rotational position of the waveplate. In some examples, the beam polarizer 226 passes or blocks the light that is polarized and rotated in order to control the amount of electromagnetic radiation included in the laser pulses output from the laser system 202. In various embodiments, the laser pulses output from the laser system 202 are optically transmitted (e.g., via an optical fiber) to a handpiece 228.

In addition to the laser 210, the optical switching device 212, and the pulse picking controller 204, the laser system 202 may have other components. For example, the laser system 202 may include components for operating the laser 210, such as a power supply, laser pumps, laser energy control, and monitor. In addition, the laser system 202 may include other components in the optical path of the laser output, such as one or more lenses, mirrors, and optical fibers. The laser system 202 is generally designed to direct laser electromagnetic radiation from the laser 210 to an output port. The laser system 202 may direct the laser electromagnetic radiation from the laser 210 to the output port through one or more optical components, such as lenses and mirrors.

An instrument may be optically connected to the laser system 202 to receive the laser electromagnetic radiation from the output port. The instrument may be, for example, the handpiece 228 which can be a handpiece for an ophthalmic procedure. The instrument or handpiece may be connected to the laser system 202 by a delivery optical fiber. The delivery optical fiber may be flexible and relatively long (e.g., 6 feet, 8 feet, 10 feet, etc.) to give an operator flexibility in maneuvering the instrument or handpiece at some distance away from the laser system 202. The laser electromagnetic radiation may be transmitted from the laser system 202, through the delivery optical fiber and the instrument or handpiece, and from an output tip of the instrument or handpiece to the desired target, such as a lens or lens fragment in the eye of a patient.

In some embodiments, the laser system 202 may be suitable for cataract surgery. In some embodiments, the output energy of the laser system 202 is suitable for fragmentation and/or emulsification of a cataractous lens. In some examples, the laser output is used for fragmentation and/or phacoemulsification of the lens to a sufficient degree for removal of the lens.

In some embodiments, the laser system 202 may be suitable for glaucoma surgery. In some embodiments, the output energy of the laser system 202 is suitable for making or facilitating the formation of a drainage channel in eye tissue. In some examples, the drainage channel is configured to reduce intraocular pressure.

The laser 210 may be any type of laser suitable for the desired application. The laser 210 may output suitable electromagnetic radiation at any suitable wavelength. For example, the laser 210 may emit electromagnetic radiation in one or more wavelengths in the visible, infrared, and/or ultraviolet wavelengths. The laser 210 may operate or be operated to emit a continuous beam of electromagnetic radiation. Alternatively, the laser 210 may operate or be operated to emit a pulsed beam.

In one or more examples, the laser 210 operates in the infrared range. For example, the laser 210 may output electromagnetic radiation in the mid-infrared range, e.g., in a range of about 2.0 microns to about 4.0 microns. Some examples of wavelengths include about 2.5 microns to 3.5 microns, such as about 2.775 microns, about 2.8 microns, or about 3.0 microns. Such a laser may be suitable, for example, for lens fragmentation in cataract surgery, or for other procedures.

In various embodiments, the optical switching device 212 is a device that operates either to allow laser electromagnetic radiation, e.g., laser pulses, emitted from the laser 210 to be output from the laser system 202 or to prevent laser electromagnetic radiation, e.g., laser pulses, emitted from the laser 210 from being output from the laser system 202. The optical switching device 212 may switch back and forth between these two conditions, under the control of the pulse picking controller 204. For example, the optical switching device 212 may include a shutter that is moved by a shutter motor into and out of the path of laser electromagnetic radiation, to selectively allow or prevent laser electromagnetic radiation from being output from the laser system 202. The shutter motor may be configured to move the shutter in an alternating manner between a first position corresponding to a first condition of the optical switching device 212 (in which it allows laser electromagnetic energy, e.g., laser pulses, emitted from the laser 210 to be output from the laser system 202) and a second position corresponding to a second condition of the optical switching device 212 (in which it prevents laser electromagnetic energy, e.g., laser pulses, emitted from the laser 210 from being output from the laser system 202). In an example, the shutter comprises a mirror, and the shutter motor comprises a galvanometer motor.

In another example, the optical switching device 212 may comprise: (i) a shutter having an axis of rotation and at least one open area and at least one solid area arranged around the axis of rotation of the shutter, and (ii) a shutter motor configured to rotate the shutter around the axis of rotation of the shutter. In such an example, the first condition of the optical switching device 212 (in which it allows laser electromagnetic energy, e.g., laser pulses, emitted from the laser 210 to be output from the laser system 202) corresponds to a position of the shutter in which a solid area of the shutter is not in a path of the laser pulses emitted from the laser 210, and the second condition of the optical switching device 212 (in which it prevents laser electromagnetic energy, e.g., laser pulses, emitted from the laser 210 from being output from the laser system 202) corresponds to a position of the shutter in which a solid area of the shutter is in the path of the laser pulses emitted from the laser 210.

The optical switching device 212 may further comprise a laser energy control system configured to regulate the amount of electromagnetic energy of each laser pulse that exits the laser system 202. For example, the laser energy control system may comprise a waveplate, a waveplate motor, and a polarizer plate, wherein the waveplate motor is configured to move the waveplate into different positions corresponding to different percentages of laser electromagnetic energy permitted to pass through the laser energy control system. In another alternative embodiment, the optical switching device 212 may comprise a pockels cell. A pockels cell optical switching device may switch back and forth, under the control of the pulse picking controller 204, between a first condition in which it allows laser pulses emitted from the laser 210 to be output from the laser system 202 and a second condition in which it prevents laser pulses emitted from the laser 210 from being output from the laser system 202. Also, a pockels cell optical switching device can be operated incrementally to allow different percentages of electromagnetic energy emitted by the laser 210 to be output by the laser system 202.

The pulse picking controller 204 may be configured to communicate optical switching control signals to the optical switching device 212. The optical switching control signals are based on inputs to the surgical system 200 (e.g., inputs to the surgical console 100), including from the input device 104, if provided. In some embodiments, the optical switching control signals may be based on inputs provided via the handpiece 228.

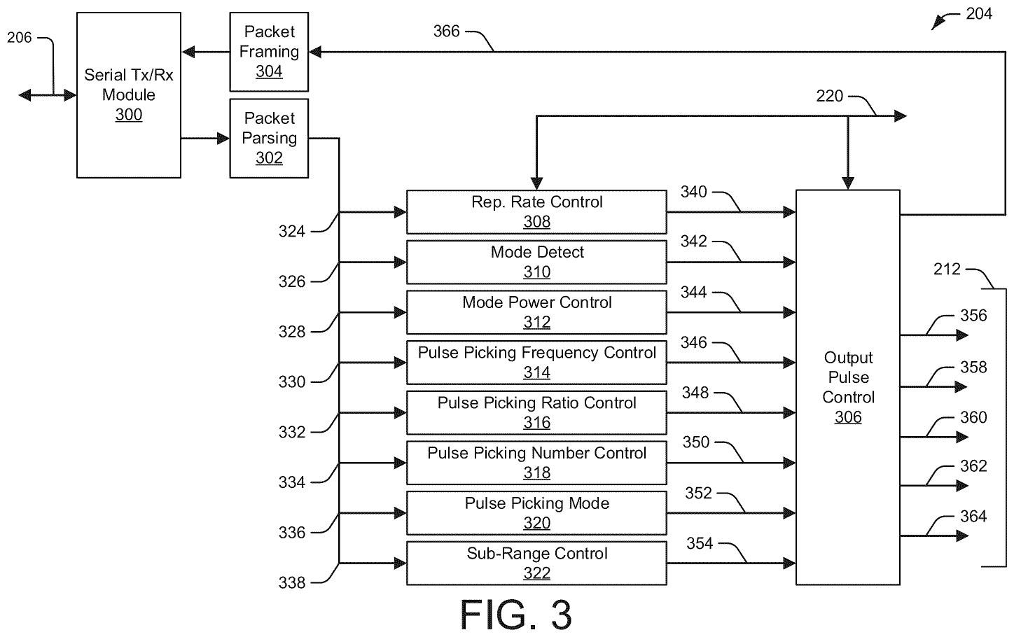

FIG. 3 illustrates an example architecture of a pulse picking controller, according to embodiments described herein. As depicted in FIG. 3, the pulse picking controller 204 includes a serial transmitter/receiver (Tx/Rx) module 300, a packet parsing module 302, a packet framing module 304, and an output pulse control module 306. In various embodiments, the pulse picking controller 204 may also include a repetition rate control module 308, a mode detect module 310, a mode power control module 312, a pulse picking frequency control module 314, a pulse picking ratio control module 316, a pulse picking number control module 318, a pulse picking mode module 320, and a sub-range control module 322. Although modules 300-322 are described as distinct modules, it is to be appreciated that the pulse picking controller 204 may include additional modules or fewer modules than illustrated in FIG. 3. It is also to be appreciated that each of the modules 300-322 may be implemented using software, firmware, hardware, or a combination of software, and/or firmware, and/or hardware.

In one or more embodiments, the Tx/Rx module 300 receives an input transmitted from the surgical console 100 (e.g., from the input device 104) via the connection 206. For example, the input is a message or a packet of data as described further below. In some embodiments, the Tx/Rx module 300 communicates the input received via the connection 206 to the packet parsing module 302. The packet parsing module 302 receives and processes the input, and the packet parsing module 302 may parse or segment the input into repetition rate data 324, mode data 326, power data 328, pulse picking frequency data 330, pulse picking duty ratio data 332, pulse picking number data 334, pulse picking mode data 336, and sub-range data 338.

In an example, the repetition rate control module 308 receives and processes the repetition rate data 324 in order to extract a repetition rate 340 described by the repetition rate data 324. The repetition rate control module 308 can communicate the repetition rate 340 to the output pulse control module 306. In one example, the mode detect module 310 receives and processes the mode data 326 in order to communicate a mode 342 (e.g., a sculpt mode, a quad mode, etc.) described by the mode data 326 to the output pulse control module 306. In another example, the mode power control module 312 receives and processes the power data 328 and extracts a power setting 344 (e.g., 0 percent to 100 percent) described by the power data 328. The mode power control module 312 may communicate the power setting 344 to the output pulse control module 306.

In an embodiment, the pulse picking frequency control module 314 processes the pulse picking frequency data 330 to extract a pulse picking frequency 346. The pulse picking frequency control module 314 may communicate the pulse picking frequency 346 to the output pulse control module 306. For instance, the pulse picking ratio control module 316 can receive and process the pulse picking duty ratio data 332 to extract a pulse picking duty ratio 348 described by the pulse picking duty ratio data 332. In an example, the pulse picking ratio control module 316 communicates the pulse picking duty ratio 348 to the output pulse control module 306.

In various embodiments, the pulse picking number data 334 describes a pulse picking number 350 (e.g., a number of laser pulses to be included in each pulse picking cycle). The pulse picking number control module 318 may receive and process the pulse picking number data 334 in order to communicate the pulse picking number 350 to the output pulse control module 306. In some embodiments, the pulse picking mode module 320 processes the pulse picking mode data 336 to extract a pulse picking mode 352 (e.g., normal, burst, etc.) described by the pulse picking mode data 336. The pulse picking mode module 320 can communicate the pulse picking mode 352 to the output pulse control module 306.

In one or more embodiments, the sub-range control module 322 receives the sub-range data 338 as describing a sub-range 354 (e.g., SubRange1, SubRange2, SubRange3, etc.). In an example in which the input device 104 is a footswitch, the footswitch can be pressed downward to various positions (e.g., sub-ranges) in order to specify the sub-range 354 described by the sub-range data 338 as described further below. The sub-range control module 322 can process the sub-range data 338 in order to communicate the sub-range 354 to the output pulse control module 306.

As illustrated in FIG. 3, the output pulse control module 306 receives the repetition rate 340, the mode 342, the power setting 344, the pulse picking frequency 346, the pulse picking duty ratio 348, the pulse picking number 350, the pulse picking mode 352, and the sub-range 354 as inputs. In various embodiments, the output pulse control module 306 also receives the laser trigger 220 as one of the inputs. In some embodiments, the output pulse control module 306 processes the inputs received in order to generate outputs which may include a mode power command 356, a repetition rate output command 358, a pulse picking duty cycle command 360, a pulse picking synchronization command 362, and a pulse picking control command 364. In one or more embodiments, the output pulse control module 306 communicates the mode power command 356, the repetition rate output command 358, the pulse picking duty cycle command 360, the pulse picking synchronization command 362, and the pulse picking control command 364 to the optical switching device 212. In an example, hollow motor 214 receives the mode power command 356 via the laser power control 222. In another example, flip mirror 216 receives the repetition rate output command 358, the pulse picking duty cycle command 360, the pulse picking synchronization command 362, and the pulse picking control command 364 via the pulse picking control 224.

In some embodiments, the output pulse control module 306 communicates a message confirmation 366 to the packet framing module 304. In various embodiments, the packet framing module 304 receives and processes the message confirmation 366 to format data included in the message confirmation 366 in a format which may be similar to the format of the inputs received by the packet parsing module 302. In an example, the packet framing module 304 formats the message confirmation 366 as packets of data for communication to the serial Tx/Rx module 300. In one or more examples, the serial Tx/Rx module 300 communicates data included in the message confirmation 366 to the surgical console 100 via the connection 206.

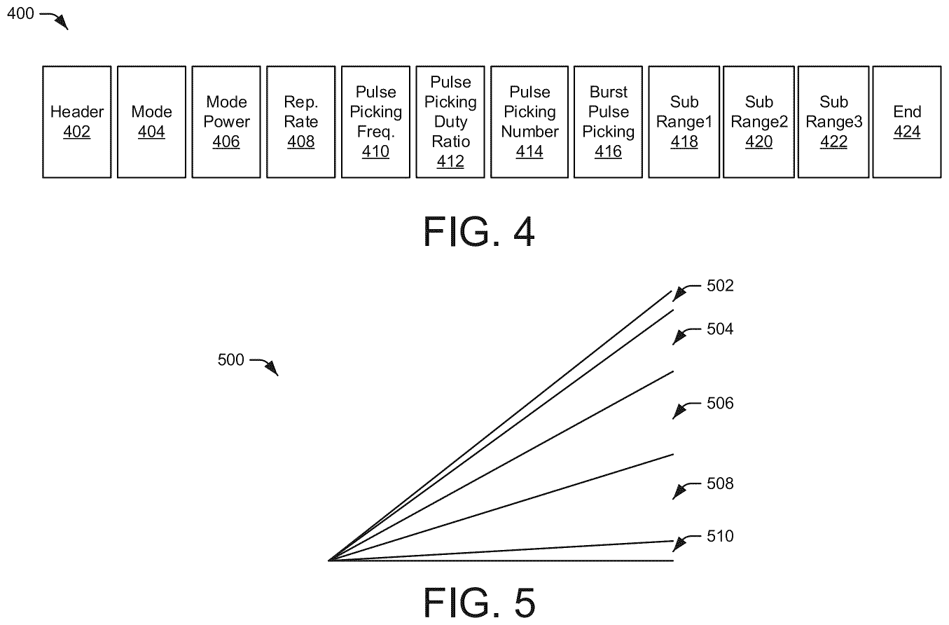

FIG. 4 illustrates an example of a message to communicate as an input to a pulse picking controller, according to embodiments described herein. As shown, the message 400 includes packets of instructions and/or data which are organized in a format that may begin with a header 402. In one or more embodiments, the serial Tx/Rx module 300 can receive the message 400 via the connection 206 from the surgical console 100. In an example, the packet parsing module 302 processes the message 400 to identify the header 402 which indicates the beginning of the message 400.

In various embodiments, a mode 404 follows the header 402, and the mode 404 indicates a selected mode of operation for the laser system 202. Examples of the mode 404 include a sculpting mode, a quad mode, other modes, etc. In an embodiment, a mode power 406 follows the mode 404, and the mode power 406 indicates an amount of electromagnetic radiation to be included in laser pulses output from the laser system 202. For example, the mode power 406 is expressed as a percentage of a total power (e.g., 0 to 100 percent).

In some embodiments, a repetition rate 408 follows the mode power 406. The repetition rate 408 can define a selected base repetition rate for the laser 210. For example, the repetition rate 408 defines a rate of laser pulses to be emitted from the laser 210. Examples of the repetition rate 408 include 1 kHz (1000 laser pulses per second), 1.5 kHz (1500 laser pulses per second), a frequency between 1 and 1.5 kHz, etc.

In one or more embodiments, a pulse picking frequency 410 follows the repetition rate 408, and the pulse picking frequency 410 can be related to the repetition rate 408. For example, the pulse picking frequency 410 defines a selected length of a pulse picking cycle. In some examples, the pulse picking frequency 410 may not be greater than the repetition rate 408. Examples of the pulse picking frequency 410 can include 1 Hz, 500 Hz, a frequency between 1 and 500 Hz, a frequency less than 1 Hz, and so forth.

In one or more embodiments, a pulse picking duty ratio 412 follows the pulse picking frequency 410 in the message 400. In various examples, the pulse picking duty ratio 412 defines a relative amount of time during a pulse picking cycle that laser pulses are emitted from the laser system 202 (e.g., 1 to 100 percent). In some examples, the pulse picking duty ratio 412 can correspond to a number of laser pulses included in a pulse picking cycle.

In various embodiments, a pulse picking number 414 follows the pulse picking duty ratio 412. In one example, the pulse picking number 414 defines a selected number of laser pulses to be emitted by the laser system 202 during a pulse picking cycle. In another example, the pulse picking number 414 defines a maximum number of laser pulses to be emitted by the laser system 202 during a pulse picking cycle.

In some embodiments, a burst pulse picking 416 follows the pulse picking number 414 in the message 400. The burst pulse picking 416 can indicate a type of pulse picking mode which has been selected (e.g., a normal mode, a burst mode, etc.). In one or more examples, the mode 404 can be set to a quad mode if the burst pulse picking 416 indicates that a type of pulse picking mode has been selected. In various embodiments, if the burst pulse picking 416 indicates a normal mode, then a synchronous firing timer (e.g., synchronous with the laser trigger 220) is selected. In one or more embodiments, if the burst pulse picking 416 indicates a burst mode, then an asynchronous firing timer (e.g., asynchronous with the laser trigger 220 and synchronous with the message 400) is selected.

In one or more embodiments, a SubRange1 418 follows the burst pulse picking 416 in the message 400. In examples in which the input device 104 is a footswitch, the input device 104 is adjustable within a first subrange, and the SubRange1 418 can indicate a position of a pedal of the input device 104 within the first subrange (e.g., 0 to 100). For example, if the SubRange1 418 has a value of 0, then the pedal has not actuated into the first subrange. In some embodiments, if the SubRange1 418 has a value of 100, then the pedal has actuated completely through the first subrange. In an example, if the SubRange1 has a value of 50, then the pedal is actuated halfway through the first subrange. In some embodiments, the first subrange is a first pre-active range (e.g., actuation of the pedal of the input device 104 within the first subrange does not energize the laser 210).

In various embodiments, a SubRange2 420 follows the SubRange1 418. The SubRange2 420 indicates a position of the pedal of the input device 104 within a second subrange (e.g., 0 to 100). Similar to the SubRange1 418, if the SubRange2 420 has a value of 0, then the pedal has not actuated into the second subrange. If the Subrange2 420 has a value of 100, then the pedal has actuated completely through the second subrange. In one or more embodiments, the second subrange is a second pre-active range (e.g., actuation of the pedal of the input device 104 within the second subrange does not energize the laser 210).

In some embodiments, a SubRange3 422 follows the SubRange2 420 in the message 400. For example, the SubRange3 422 indicates a position of the pedal of the input device 104 within a third subrange (e.g., 0 to 100). In an example, the third subrange is an active subrange, and an actuation of the pedal of the input device 104 within the third subrange energizes the laser 210. In one or more embodiments, an end 424 follows the SubRange3 422. For example, the packet parsing module 302 processes the message 400 to identify the end 424 which indicates an end of the message 400. In other examples, one or more additional operational subranges follow the SubRange3 422. For example, a SubRange 4, SubRange 5, SubRange 6, etc., may follow SubRange 3 422.

FIG. 5 illustrates an example of operational subranges for an input device, according to embodiments described herein. As depicted in FIG. 5, example 500 includes a range zero 502, a range one 504, a range two 506, a range three 508, and a bottom range 510. As described above, the pedal of the input device 104 can actuate within the range one 504 (the first subrange), the range two 506 (the second subrange), and the range three 508 (the third subrange). In various embodiments, an actuation of the pedal of the input device 104 into the range one 504 activates the surgical console 100 for a specific function, such as irrigation, without energizing the laser 210. In some embodiments, an actuation of the pedal of the input device 104 into the range two 506 activates the surgical console 100 for a different specific function, such as aspiration, without energizing the laser 210. For example, the irrigation function may or may not continue operation while the pedal of the input device 104 is actuated within the range two 506. In one or more embodiments, an actuation of the pedal of the input device 104 into the range three 508 energizes the laser 210. In various examples, the irrigation and/or the aspiration may or may not continue operation while the pedal of the input device 104 is actuated into the range three 508. By adjusting or actuating the pedal of the input device 104 within the range three 508, an operator may dynamically adjust output from the laser system 202. In some embodiments, adjusting or actuating the pedal of the input device 104 within the range three 508 specifies a value of the SubRange3 422 which can be used to specify values of the mode power 406, the repetition rate 408, the pulse picking frequency 410, the pulse picking duty ratio 412, and/or the pulse picking number 414. Note that although three operational subranges are shown, an input device as described herein may have more or less operational subranges.

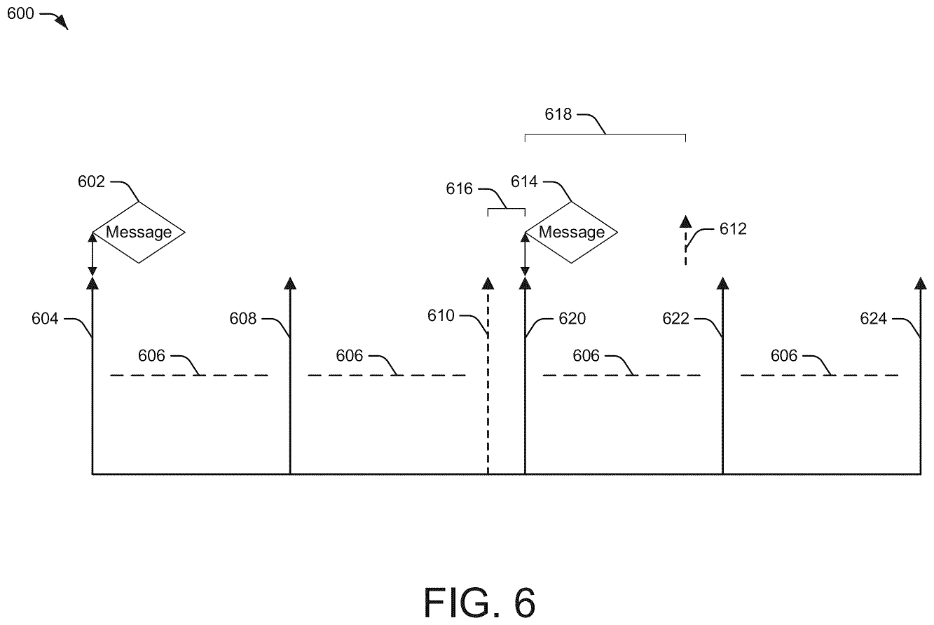

FIG. 6 illustrates an example representation of selecting an asynchronous firing timer, according to embodiments described herein. As shown, the representation 600 includes a first message 602 (e.g., a first input received via the connection 206). In some embodiments, the first message 602 has a same content and format as the message 400. For example, the repetition rate 408 of the first message 602 is 1 kHz; the pulse picking frequency 410 of the first message 602 is 1 Hz; and the pulse picking number 414 of the first message 602 is 1000. As shown, the first message 602 is synchronized with the laser trigger 220 such that the first message 602 is aligned with a beginning 604 of a first pulse picking cycle that outputs 1000 laser pulses 606 from the laser system 202. In an embodiment, a synchronous firing timer is selected for controlling the flip mirror 216 since the first message 602 is synchronized with the base repetition rate (e.g., 1 kHz) of the laser 210. For example, the synchronous firing timer causes the beginning 604 of the first pulse picking cycle to start based on the base repetition rate of the laser system 202 (e.g., the base repetition rate of the laser 210).

In the illustrated example, the first pulse picking cycle ends at a beginning 608 of a second pulse picking cycle which also outputs 1000 laser pulses 606 from the laser system 202. For instance, the second pulse picking cycle ends at an expected beginning 610 of a next pulse picking cycle. In some examples, the next pulse picking cycle could output 1000 laser pulses from the laser system 202, and then the next pulse picking cycle may end at an expected beginning 612 of an additional next pulse picking cycle. However, as illustrated in the representation 600, a second message 614 is received (e.g., a second input received via the connection 206). In various embodiments, the second message 614 may have a same content and format as the message 400. For example, the pulse picking frequency 410 of the second message 614 is 1 Hz and the pulse picking number 414 of the second message 614 is 1000.

In an example, the second message 414 is not synchronized with the with the base repetition rate of the laser 210. Instead, the second message 614 lags behind the expected beginning 610 of the next pulse picking cycle by a delay 616. In some examples, because of the delay 616, if the synchronous firing timer is selected for controlling the flip mirror 216, then a next pulse picking cycle does not begin until the expected beginning 612 of the additional next pulse picking cycle. This would result in an undesirable latency 618 between an expected output of 1000 laser pulses 606 and an actual output of 1000 laser pulses 606.

In order to avoid or mitigate the latency 618 in some examples, an asynchronous firing timer is selected for controlling the flip mirror 216. In one or more embodiments, the asynchronous firing timer can be utilized to realign the laser trigger 210 with the second message 614 such that a beginning 620 of the next pulse picking cycle is synchronized with the second message 614. In some examples, the next pulse picking cycle outputs 1000 laser pulses 606 from the laser system 202. For example, the next pulse picking cycle ends at a beginning 622 of an additional pulse picking cycle. The additional pulse picking cycle outputs 1000 laser pulses 606 from the laser system 202, and the additional pulse picking cycle ends at a beginning 624 of another pulse picking cycle.

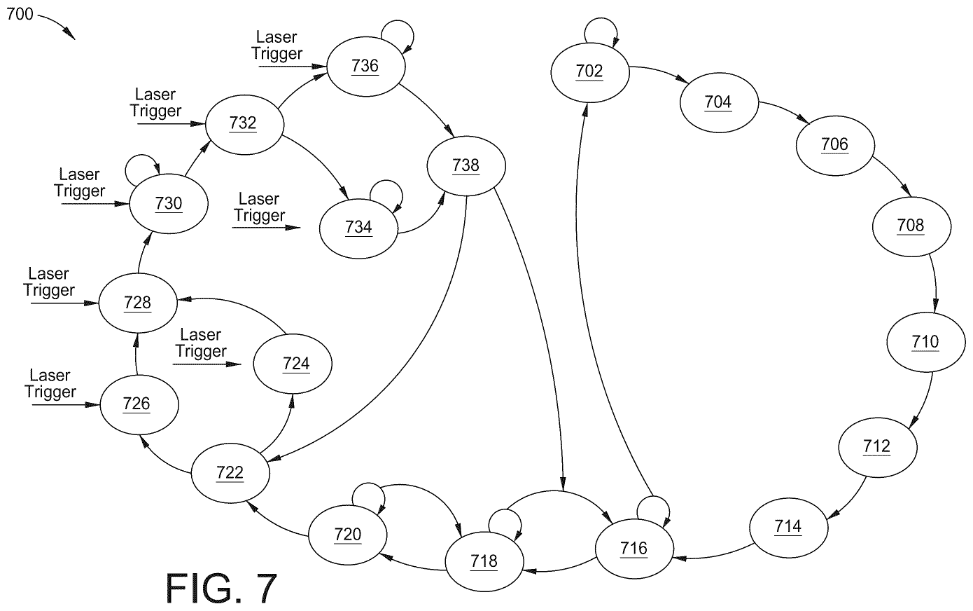

FIG. 7 illustrates an example flow diagram for selecting a synchronous firing timer or an asynchronous firing timer, according to embodiments described herein. As depicted in FIG. 7, the flow diagram 700 begins with a mode state 702. In an embodiment, while in the mode state 702, the serial Tx/Rx module 300 monitors the connection 206 for receipt of a message 400, and the packet parsing module 302 attempts to identify a header 402 which indicates a beginning of the message 400. For example, while in the mode state 702, the packet parsing module 302 parses through the message 400 to identify a mode 404 following the header 402.

In various embodiments, if the message 400 is received, and if the mode 404 indicates a selected mode of operation for the laser system 202, then the mode state 702 can proceed to a mode power state 704. While in the mode power state 704, the packet parsing module 302 parses through the message 400 to identify a mode power 406 following the mode 404. In one or more embodiments, if the packet parsing module 302 identifies the mode power 406 in the message 400, then the mode power state 704 may proceed to a repetition rate state 706. While in the repetition rate state 706, the packet parsing module 302 parses through the message 400 to identify a repetition rate 408 following the mode power 406.

In an example in which the packet parsing module 302 identifies the repetition rate 408 in the message 400, then the repetition rate state 706 can proceed to a pulse picking frequency state 708. While in the pulse picking frequency state 708, the packet parsing module 302 parses through the message 400 in order to identify a pulse picking frequency 410 following the repetition rate 408. If the packet parsing module 302 identifies the pulse picking frequency 412 in the message 400, then the pulse picking frequency state 708 may proceed to a pulse picking duty ratio state 710. While in the pulse picking duty ratio state 710, the packet parsing module 302 parses the message 400 in order to identify a pulse picking duty ratio 412 following the pulse picking frequency 410.

In some examples, if the packet parsing module 302 identifies the pulse picking duty ratio 412 in the message 400, then the pulse picking duty ratio state 710 may proceed to a pulse picking number state 712. While in the pulse picking number state 712, the packet parsing module 302 parses the message 400 in order to identify a pulse picking number 414 following the pulse picking duty ratio 412. For example, if the packet parsing module 302 identifies the pulse picking number 414 in the message 400, then the pulse picking number state 712 can proceed to a pulse picking mode state 714. While in the pulse picking mode state 714, the packet parsing module 302 parses the message 400 in order to identify a burst pulse picking 416 following the pulse picking number 414. For instance, if the packet parsing module 302 identifies the burst pulse picking 416 in the message, then the pulse picking mode state 714 may proceed to a SubRange 1 state 716.

From the SubRange 1 state 716, if the SubRange1 418 has a value of 0, then the SubRange 1 state 716 proceeds to the mode state 702. In general, if the SubRange1 418 has the value of 0, then this indicates that a user of the surgical console 100 has not actuated the pedal of the input device 104 or that the user of the surgical console 100 has not actuated the pedal of the input device into the range one 504 (e.g., the user of the surgical console 100 has not actuated the pedal of the input device 104 through the range zero 502). For example, if the pedal of the input device 104 has not actuated into the range one 504 (the first subrange), then the SubRange 1 state 716 proceeds to the mode state 702, and the packet parsing module 302 parses through the message 400 to identify the mode 404 following the header 402. If the SubRange1 418 has a value in a range of 1 to 99, then the flow diagram 700 remains at the SubRange 1 state 716. Generally, if the SubRange1 418 has the value in the range of 1 to 99, then this indicates that the user of the surgical console 100 has actuated the pedal of the input device 104 such that the pedal is positioned within the range one 504. In an example, if the pedal of the input device 104 is positioned within the range one 504, then the SubRange 1 state 716 does not change. If the SubRange1 418 has a value of 100, then the SubRange 1 state 716 can proceed to a SubRange 2 state 718. In general, if the SubRange1 418 has the value of 100, then this indicates that the user of the surgical console 100 has actuated the pedal of the input device 104 through the range one 504 (e.g., the user of the surgical console 100 has actuated the pedal of the input device 104 past the range one 504). In one example, if the pedal of the input device 104 has actuated through the range one 504, then the SubRange 1 state 716 proceeds to the SubRange 2 state 718.

For example, from the SubRange 2 state 718, if the SubRange2 420 has a value of 0, then the SubRange 2 state 718 proceeds to the SubRange 1 state 716. Generally, if the SubRange2 420 has the value of 0, then this indicates that the user of the surgical console 100 has actuated the pedal of the input device 104 such that the pedal is not positioned within the range two 506 or past the range two 506. In some examples, if the pedal of the input device 104 has actuated upwardly and out from the range two 506 (the second subrange), then the SubRange 2 state 718 proceeds to the SubRange 1 state 716. If the SubRange2 420 has a value in a range of 1 to 99, then the flow diagram 700 remains at the SubRange 2 state 718. In general, if the SubRange2 420 has the value in the range of 1 to 99, then this indicates that the user of the surgical console 100 has actuated the pedal of the input device 104 such that the pedal is positioned within the range two 506. For example, if the pedal of the input device 104 is positioned within the range two 506, then the SubRange 2 state 718 does not change. If the SubRange2 420 has a value of 100, then the SubRange 2 state 718 may proceed to a SubRange 3 state 720. Generally, if the SubRange2 420 has the value of 100, then this indicates that the user of the surgical console 100 has actuated the pedal of the input device 104 through the range two 506 (e.g., the user of the surgical console 100 has actuated the pedal of the input device 104 past the range two 506). In one or more examples, if the pedal of the input device 104 has actuated through the range two 506, then the SubRange 2 state 718 proceeds to the SubRange 3 state 720.

From the SubRange 3 state 720, if the SubRange3 422 has a value of 0, then the SubRange 3 state 720 proceeds to the SubRange 2 state 718. In general, if the SubRange3 422 has the value of 0, then this indicates that the user of the surgical console 100 has actuated the pedal of the input device 104 such that the pedal is not positioned within the range three 508 or past the range three 508. In an example, if the pedal of the input device 104 has actuated upwardly and out from the range three 508 (the third subrange), then the SubRange 3 state 720 proceeds to the SubRange 2 state 718. If the SubRange3 422 has a value in a range of 1 to 100, then the SubRange 3 state 720 can proceed to a burst picking mode state 722. Generally, if the SubRange3 422 has the value in the range of 1 to 100, then this indicates that the user of the surgical console 100 has actuated the pedal of the input device 104 such that the pedal is either positioned within the range three 508 or within the bottom range 510. For example, if the pedal of the input device 104 is positioned within the range three 508, then the SubRange 3 state 720 proceeds to the burst picking mode state 722.

At the burst picking mode state 722, if the burst pulse picking 416 indicates a normal mode, then the flow diagram 700 proceeds to a normal mode state 724 and the synchronous firing timer is selected. In various examples, at the burst picking mode state 722, if the burst pulse picking 416 indicates the normal mode, then the pulse picking controller 204 selects the synchronous firing timer to cause a beginning of a pulse picking cycle to start based on a base repetition rate (e.g., the repetition rate 408) of the laser system 202. At the burst picking mode state 722, if the burst pulse picking 416 indicates a burst mode, then the flow diagram 700 proceeds to a burst mode state 726 and the asynchronous firing timer is selected. In some examples, at the burst picking mode state 722, if the burst pulse picking 416 indicates the burst mode, then the pulse picking controller 204 selects the asynchronous firing timer to cause the beginning of the pulse picking cycle to start based on a receipt of an input (e.g., the message 400).

For example, the asynchronous firing timer is configured to mitigate a latency between the receipt of the input (e.g., the message 400) and switching the optical switching device 212 to a condition which allows laser pulses emitted by the laser 210 to be output from the laser system 202. In an example, the asynchronous firing timer is configured to mitigate the latency if the input (e.g., the message 400) is received before an expected start of the beginning of the pulse picking cycle based on the base repetition rate (e.g., the repetition rate 408) of the laser system 202. In one example, the asynchronous firing timer is configured to mitigate the latency if the pulse picking cycle has a pulse picking frequency (e.g., the pulse picking frequency 410) of 10 Hz or less.

From either the normal mode state 724 or the burst mode state 726, the flow diagram 700 proceeds to a flip mirror on state 728 (e.g., the flip mirror 216 allows the laser pulse train 218 to be output from the laser system 202), then to a pulse count state 730, and then to a flip mirror off state 732 (e.g., the flip mirror 216 prevents the laser pulse train 218 from being output from the laser system 202). For example, from the flip mirror off state 732, if the burst pulse picking 416 indicates a normal mode, then the flow diagram 700 proceeds to a normal mode state 734 and the synchronous firing timer is selected. In some examples, at the flip mirror off state 732, if the burst pulse picking 416 indicates the normal mode, then the pulse picking controller 204 selects the synchronous firing timer. In an example, from the flip mirror off state 732, if the burst pulse picking 416 indicates a burst mode, then the flow diagram 700 proceeds to a burst mode state 736 and the asynchronous firing timer is selected. In various examples, at the flip mirror off state 732, if the burst pulse picking 416 indicates the burst mode, then the pulse picking controller 204 selects the asynchronous firing timer.

From either the normal mode state 734 or the burst mode state 736, the flow diagram proceeds to a SubRange3 state 738. For instance, from the SubRange3 state 738 if the SubRange3 422 has a value of 0, then the SubRange 3 state 738 may proceed to the SubRange 2 state 718. In one example, at the SubRange3 state 738 if the pedal of the input device 104 has actuated upwardly and out from the range three 508, then the SubRange 3 state 738 proceeds to the SubRange 2 state 718. From the SubRange3 state 738 if the SubRange3 422 has a value of greater than 0, then the SubRange 3 state 738 may proceed to the burst picking mode state 722. In some examples, at the SubRange3 state 738 if the pedal of the input device 104 is positioned within the range three 508, then the SubRange 3 state 738 proceeds to the burst picking mode state 722.

The above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. A laser system comprising:
a laser configured to emit electromagnetic radiation in laser pulses;
an optical switching device configured to switch between a first condition that allows laser pulses emitted by the laser to be output from the laser system and a second condition that prevents the laser pulses emitted by the laser from being output from the laser system; and
a pulse picking controller configured to:
receive inputs from an input device;
communicate optical switching control signals to the optical switching device based on the inputs; and
select at least one of a synchronous firing timer or an asynchronous firing timer, the asynchronous firing timer causes a beginning of a pulse picking cycle to start based on receipt of an input of the inputs.

2. The laser system of claim 1, wherein the synchronous firing timer causes the beginning of the pulse picking cycle to start based on a base repetition rate of the laser system.

3. The laser system of claim 1, wherein the pulse picking controller selects the synchronous firing timer or the asynchronous firing timer based on a pulse picking mode indicated by the input.

4. The laser system of claim 1, wherein the asynchronous firing timer is configured to mitigate latency between the receipt of the input and switching the optical switching device to the first condition.

5. The laser system of claim 1, wherein the pulse picking controller selects the asynchronous firing timer based on the input and wherein the pulse picking controller selects the synchronous firing timer based on receipt of an additional input of the inputs.

6. The laser system of claim 5, wherein the input indicates a first pulse picking mode and the additional input indicates a second pulse picking mode.

7. The laser system of claim 1, wherein the input is received before an expected start of the beginning of the pulse picking cycle based on a base repetition rate of the laser system.

8. The laser system of claim 7, wherein the pulse picking controller selects the asynchronous firing timer.

9. The laser system of claim 1, wherein the input device includes an adjustable footswitch configured to actuate within subranges to communicate the inputs to the pulse picking controller.

10. The laser system of claim 1, wherein the pulse picking cycle has a pulse picking frequency of 10 hertz or less.

11. A method comprising:
emitting laser pulses by a laser of a laser system, the laser configured to emit electromagnetic radiation in laser pulses;
receiving inputs from an input device;
controlling an optical switching device based on the inputs, the optical switching device configured to switch between a first condition that allows the laser pulses emitted by the laser to be output from the laser system and a second condition that prevents the laser pulses emitted by the laser from being output from the laser system; and
selecting at least one of a synchronous firing timer or an asynchronous firing timer, the asynchronous firing timer configured to cause a beginning of a pulse picking cycle to start based on receipt of an input of the inputs.

12. The method of claim 11, wherein the synchronous firing timer is configured to cause the beginning of the pulse picking cycle to start based on a base repetition rate of the laser system.

13. The method of claim 11, wherein the synchronous firing timer or the asynchronous firing timer is selected based on a pulse picking mode indicated by the input.

14. The method of claim 11, further comprising controlling at least one of a length of the pulse picking cycle, a number of the laser pulses included in the pulse picking cycle, a pulse picking mode of the laser system, or an amount of the electromagnetic radiation included in the laser pulses.

15. The method of claim 11, wherein the pulse picking cycle has a pulse picking frequency of 10 hertz or less.
